(19) United States
(12) Patent Application Publication
(10) Pub. No.: US 2025/0381508 A1
(43) Pub. Date: Dec. 18, 2025

(54) AIR FILTER WITH INTEGRATED SENSORICS

(71) Applicant: KAPPA FILTER SYSTEMS GMBH, Steyr-Gleink (AT)

(72) Inventors: Klaus KRÜGER, Steyr/St. Ulrich (AT); Urs HUNZIKER, Meilen (CH)

(73) Assignee: KAPPA FILTER SYSTEMS GMBH, Steyr-Gleink (AT)

(21) Appl. No.: 18/877,397

(22) PCT Filed: Jun. 28, 2023
(86) PCT No.: PCT/EP2023/067726

§ 371 Date: Dec. 20, 2024

(30) Foreign Application Priority Data
Jul. 1, 2022 (DE) 10 2022 116 532.9

(51) Int. Cl. B01D 46/00; B01D 46/02; B01D 46/42; B01D 46/44; B01D 46/46; B01D 46/52; B01D 46/54; F24F 8/108; G01N 33/00

(52) U.S. Cl. CPC B01D 46/0086; B01D 46/023; B01D 46/429; B01D 46/442; B01D 46/444; B01D 46/446; B01D 46/448; B01D 46/46; B01D 46/521; B01D 46/543; F24F 8/108; G01N 33/0073; B01D 2279/50

(57) ABSTRACT

The present disclosure relates to a filter module for filtering air of at least a part of a building or air from an exhaust air purification unit of a production process, wherein the filter module has a filter body, which filters the air flowing through from air accompanying substances, and at least one sensor with sensor electronics. The sensor is configured for measuring a value of an analysis parameter for the analysis of at least one of the air accompanying substances and of the air quality of the air. The filter body and the sensor are configured and arranged with respect to one another in the case of an air flow through the filter module, the value of the analysis parameter which is measured with the sensor corresponds to more than 90% of the value of the analysis parameter which is averaged over the entire air flow.

### TECHNICAL FIELD

The present invention relates to a filter module and a method for filtering air of at least a part of a building or of air of an exhaust air purification unit of a production process by means of at least one sensor with sensor electronics. Furthermore, the invention relates to a filter system with the filter module.

### BACKGROUND OF THE INVENTION

Ventilation systems are used for the ventilation and venting of rooms in buildings and contain filter systems for filtering pollutants from the air. In the filter systems there are sensors which measure the quality of the air and the air accompanying substances which are carried along in it. The increasingly rapidly changing requirements for measuring tasks of the sensors in ventilation systems are difficult to implement, since ventilation systems have a service life of 10 to 30 years and their planning usually starts two years before the initial operation. The increasingly higher requirements for accuracies of measured values with respect to data from the air flow make it increasingly difficult to integrate sensor systems into ventilation systems which meet the future measuring requirements over a long operating time and are low in maintenance or even maintenance-free.

In addition, the internal flow conditions change with increasing age of ventilation systems, because the inner surfaces which are bare at the installation time are covered with a biofilm or with solid deposits. This has the result that in particular small sensors do not have the same operating conditions for the measurements over the entire service life of a system. Above all in the case of pipe bends, these problems arise in the downstream regions. Since the initially intended cleaning of inner surfaces of pipes is very rarely carried out in ventilation systems, zones of air vortices can shift due to dust deposits and biofilm formation and thus falsify measured values.

### SUMMARY OF THE INVENTION

It is an object of the present invention to provide a filter solution which permits an exact analysis of the air to be filtered over the entire service life of a ventilation system.

This object is achieved with a filter module and a method for filtering air of at least a part of a building or of air of an exhaust air purification unit of a production process according to the subject matter of the independent patent claims.

According to a first aspect, a filter module for filtering air of at least a part of a building or of air of an exhaust air purification unit of a production process is described. The filter module comprises a filter body, which filters the air which is flowing through from air accompanying substances, and at least one sensor with sensor electronics. The sensor is configured for measuring a value of an analysis parameter for the analysis of the air accompanying substances and/or of the air quality of the air. The filter body and the sensor are configured and arranged with respect to one another in such a way that, in the case of an air flow through the filter module, the value of the analysis parameter which is measured with the sensor corresponds to more than 90% to the value of the analysis parameter which is averaged over the entire air flow. The filter body is configured in such a way that a pressure drop over the filter body is less than 2500 Pascal in the case of an air volume per square meter of filter area and hour of less than 600 m³/(m²×h) and a velocity of the volume flow is in the range of 0.1 m/s to 5 m/s.

According to a further aspect, a filter system is described, which comprises a control unit and at least one above-described filter module, wherein the at least one filter module is coupled to the control unit for the exchange of analysis data, wherein the at least one filter module is coupled to the control unit for the exchange of analysis data relating to the analysis of the air accompanying substances and/or of the air quality of the air.

According to a further aspect, a method for filtering air of at least a part of a building or of air of an exhaust air purification unit of a production process with a above-described replaceable filter module is described.

A filter system according to the invention is typically used in buildings for filtering and purifying air or else for purifying air in production processes of factories. For this purpose, a filter system comprises, for example, active flow generators, such as, for example, fans, or is integrated into a ventilation system of a building, which comprises, for example, a central active flow generator.

The filter system comprises, for example, a housing, in which a filter module is arranged or a multiplicity of filter modules are arranged in series along the flow direction of the air through the filter system or parallel to the flow direction.

The filter module comprises, for example, a two-dimensional (German: flächig) filter material, which is fixed in a circumferential support frame. The filter module can be formed as a pocket filter, wherein a multiplicity of pockets of filter material are fastened in the support frame and the air flow is introduced into the pockets in order to filter the inflowing air. Furthermore, the filter module can likewise be formed as a cartridge filter, hose filter, candle filter, compact filter and HEPA filter.

The filter module according to the invention and in particular the filter material is configured in such a way that, in the case of a velocity of the volume flow of 0.1 m/s to 5 m/s through the filter body, the pressure drop of the air flowing through the filter body is less than 2500 Pascal in the case of an air volume per square meter of filter area per hour of less than 600 m³/(m²×h). Accordingly, the filter module serves for purifying large air masses with a low pressure loss. These values can be set structurally in particular by the selection of the filter material and the corresponding pore sizes and fabric structures of the filter material.

The filter module comprises in particular the filter body with a filter region which assumes the function of filtering the air. According to the invention, the sensor is arranged relative to the filter region in such a way that, in the case of a pressure drop range of 10 Pa to 450 Pa, in particular up to 250 Pa or up to 150 Pa, the composition of the air flow at the sensor changes hardly or less than 40% over the filter module compared with the composition in the filter region, and wherein the sensor is configured relative to the filter region in such a way that the air in the analysis region comes into contact with more than 90% with the same air accompanying substances or air particles as in the filter region. The filter performance of the filter module according to the invention, in particular of the filter region, is measured for example according to EN ISO 16890, and is better than 50% for one of the classes “ISO Coarse”, “ISO ePM10”, “ISO ePM2,5” or “ISO ePM1”.

If the filter module is operated within these characteristic values, the sensor can come into contact with more than 90% with the same air accompanying substances and/or air quantities as the filter body due to the arrangement of the sensor and the filter body proposed in the invention.

This configuration is achieved in particular if the sensor is arranged and configured at a suitable position in a suitable size in the filter body or the filter module. For example, the sensor comprises a sufficient distance from the support frame of the filter module to a flow channel or to the edge of a flow channel, in which the filter module is arranged in the filter system, in order thus to avoid edge flow properties which cause a different composition of the air accompanying substances or air particles of the air or a different pressure drop range of the air, relative to a, for example central, filter body. Correspondingly, the sensor is arranged for example at a distance of more than 0.5 cm, more than 1 cm, in particular of more than 2 cm from an edge region or the outer air flow boundary of the filter body.

The solution according to the Invention is suitable for filter modules, for example in the manner of a pocket filter, or secondary filter systems. With a secondary filter system, an air circulation system with filtering for installation in the room is generally described. The secondary filter system can be mobile or stationary. In contrast thereto, for example, controlled domestic ventilation, fixedly installed and piped ventilation systems are referred to as primary filter system. In the secondary filter systems, zones with laminar air flow can be created in which a corresponding filter module according to the invention is arranged. For example, a support frame of the filter material of the filter module forms a suitable mechanical strength platform, so that the sensors can be fastened directly or indirectly in a sufficiently vibration-free manner (so that no element flutter in the air flow). This reduction of vibrations is especially important when sensors which are sensitive to vibrations (for example MEMS or other electromechanical components) are used in the analysis region.

On account of the arrangement of the sensor, the filter module according to the invention in particular provides integrated support for online or offline analysis of the pollutant load of the air which is flowing through. In particular due to the arrangement of the sensor, the latter can, for example, determine measured values from the air which are representative of the air flow, in particular in a temporal or quantitative informative capacity.

The sensor with its sensor electronics is configured to carry out qualitative and/or quantitative measurements and analyses of the air and of air accompanying substances, i.e. air particles or gaseous substances. The direct measurement of air accompanying substances or groups of air accompanying substances in the air flow can be provided by means of the integrated sensor. This can relate, for example, to the amount of fine dust of a specific diameter class. Furthermore, for example, other foreign substances can be filtered away beforehand, with the result that only the specific air accompanying substances strike the sensor. In the case of turbulence due to turbulent flows of the air through the filter module, heavier substances (particles, molecules, aerosols, etc.) are moved away by centrifugal forces in the radial direction of a flow roller, which leads to a dehomogenization of the air flow composition. By means of the air module according to the invention, by ensuring the flow according to the invention, despite possible pressure differences, a representative air flow composition can be measured through the sensor, even over the entire life cycle of the filter module.

In conventional approaches, the flow conditions in a filter system are frequently unstructured, that is to say, until now, it has not been important to precisely constipate the flow conditions in the interior of a pocket filter, for example, since it only had to filter. Regions of the filter region which are poorly placed in terms of flow are occupied later when the regions through which the flow is good are already occupied and the flow resistance thereof increases as a result. The solution according to the invention ensures that the value which is measured by the sensor substantially also corresponds to the average value over the entire air flow. An exemplary measure for a homogeneous measurement is the prevention of air vortices in front of the sensor (for example by placing the sensor away from the edge of the air flow and/or by means of guide plates for flow homogenization, etc.) since, for example, solids, molecules heavier than air or aerosols are conveyed away in the radial direction of the vortex by air vortices. The sensor is therefore arranged at regions where undesired vortex formation of the air flow is reduced or eliminated.

According to a further exemplary embodiment, the sensor is configured to measure an analysis parameter of a gas, a liquid and/or a solid as an air accompanying substance and/or an air constituent, such as the CO₂ concentration in the air, for example. The analysis parameter defines in particular the chemical and/or physical properties of the air accompanying substance, with the result that an energy consumption, a savings potential, an air load and/or a CO2 footprint can be determined taking into account the analysis parameters.

According to a further exemplary embodiment, the following values of the air which is flowing through the filter body can be detected by way of example by the sensors which are integrated into the filter module: temperature, humidity, flow rate, air quantity, dew point, portion of an air accompanying substance with a specific property. By means of the sensor, gas portions, liquid portions or solids in the air flow can be detected and, for example, the chemical/physical properties thereof, in particular quantities and/or (for example average) diameters, can be determined. On account of these basic measured values, it is also possible to calculate subsequent calculations such as a CO2 footprint, (energy) savings potential (for example depending on the pressure drop across the filter, which can be influenced by the filter material or filter replacement) or energy consumption.

In particular, a virus load of the air to be filtered can be detected by at least one sensor. For example, a concentration of viruses, such as Sars-CoV 2 viruses, for example, can be determined. In this case, a biosensor is arranged in the filter module. The air to be filtered flows over the biosensor. The biomarker can comprise, for example, biomarkers which react with the viruses and cause corresponding measurable (for example optical) reactions.

The biosensor can function on the basis of the PCR test methodology (real-time quantitative reverse transcriptase polymerase chain reaction), according to which gene sequences of a virus, for example Sars-CoV 2 virus, are detected. Furthermore, the biosensor can function in the manner of an antigen test and can implement fluorescence- or chemiluminescence-based test methods in which, for example, the virus protein is detected on the basis of a specific dyeing.

In one embodiment of the biosensor, the latter can be configured as a waveguide interferometer. Such a photonic biosensor recognizes various light-based phenomena of the viruses for the rapid detection and quantification of viruses or corresponding biomarkers, Among the various photonic biosensors, silicon-photonic biosensors which are based on the principle of evanescent corrugations can be used.

Furthermore, the biosensor can be formed as a nanophotonic biosensor on the basis of interferometric bimodal waveguides (BiMWs). In order to capture and detect viruses from a sample, the surface of the BiMW sensor is modified with specific receptors which target external antigens of the virus, such as the spike(S) protein of SARS-CoV-2, for example. As soon as the air to be filtered flows over the biosensor, the virus particles are captured by the receptors on the sensor surface and generate an interferometric signal which can be recorded in real time. The reaction of the sensor is directly proportional to the virus concentration in the air to be filtered, for example, and therefore allows an exact quantification of the virus load in the air.

According to a further exemplary embodiment, the filter material of the filter body comprises a fleece, wherein the fleece is configured with one layer, preferably with multiple layers. The filter body is arranged in the filter module in particular exchangeably, wherein the fleece is formed in particular as a disposable filter. A fleece consists of fibers of limited length, continuous fibers (filaments) or cut yarns which are joined together and connected to form a fleece (a fiber layer, a fiber web). As a result of the interlinking of the fibers, an air-permeable material with narrow, small-pore air passages is provided, as a result of which a good filter effect, in particular of air particles, is achieved.

According to a further exemplary embodiment, the filter module is exchangeably arrangeable in a filter system, wherein in particular the filter body is a disposable filter. The filter module comprises in particular an information element which emits a signal regarding the due time of an exchange of the filter module.

According to the exemplary embodiment, a filter module according to the invention is exchangeably arranged in the filter system. For example, corresponding guide rails can be provided, along which the filter module can be pushed into the operating position within the filter system. Furthermore, for example, releasable fastening means, such as for example screws or clamping closures, can be provided in order to arrange the filter module modularly and exchangeably in the filter system.

By displacing at least parts of the sensorics into the regularly exchanged filter module, a filter system can be constructed in such a way that the sensors can also be replaced over the entire service life of the filter system by means of exchanging the filter module and correspondingly worn, polluted or defective sensors can be easily exchanged together with the filter module. The present invention thus provides air filters, in particular in the form of pocket filters, which comprise integrated sensors for determining parameters of the air flowing through. The system formed in this way makes it possible, in the context of the filter module change, at the same time to exchange corresponding sensor components, which are for example precalibrated and thus no calibration or initial gauging has to be made in the filter system. Thus, in the case of a filter system, for example, sensor cleaning, advance maintenance and recalibration of sensors can be dispensed with.

For example, a moisture sensor can deviate by up to 5% in the case of 20 years of aging. Within one year, the accuracy of the measurement remains virtually constant, or deteriorates by less than 0.25%. Therefore, in the case of an annual exchange of the filter module together with sensor, a high measurement accuracy can be kept high over the life cycle of a filter system.

Since an exchangeable filter module (in particular as a disposable filter) cannot be adapted exactly to the surrounded housing of the filter system, it is furthermore advantageous if the filter module prevents possible air resonances. In the case of filter materials composed of regularly arranged filter medium (for example woven, punched, etched or drilled filters), there is the possibility that resonances and therefore negative effects arise as a result of self-organizing effects of the air flow (noises, detachment again of already embedded pollutants, in particular during start-up and stop of the system, in the case of variance of physical measured values, etc. It has been found that, in the case of the solution according to the invention, the use of a layer of a fleece damps this vibration effect. This damping arises as a result of fibers being deposited irregularly and randomly and being brought into adhesion. This irregularity reduces the vibration-related self-organization potential. This damping can be intensified when using multiple fleece layers in the construction of the filter material, in particular if these comprise at least slightly different fleece materials or fleece layers. A difference can be generated by the production of fleece materials.

According to a further exemplary embodiment, the sensor is arranged on an exhaust air side of the filter body. The placement of the sensor on the supply air side of the filter system can lead to an occupation of the sensor with foreign substances, which in turn leads to measurement errors or a slowed reaction to measurement criteria (for example, dust on a temperature sensor leads to an insulation, which above all also disturbs the measurement dynamics). This can be solved by the sensor being placed on the exhaust air side of the filter system.

According to a further exemplary embodiment, the sensor can be operated discontinuously, in particular in a duty cycle of less than 1:10, in particular in a duty cycle of less than 1:100. A duty cycle of 1:10 means, for example, that of 10 time units in which the filter region is flowed through, 1 time unit flows against or is flown through the sensor. A discontinuous measurement is therefore made possible, in particular in order to save energy for the measurement system. For example, it may be sufficient for the measurement of the sensor to be able to be carried out only during a very short time with a long rest phase, Specifically, foreign material loads in an air flow usually occur over a relatively long time. Thus, temporal intermediate values can also be interpolated from individual measured values without a continuous measurement having to be carried out. Thus, it has been possible to achieve good measurement results with a duty cycle of less than 1:10, in particular in a duty cycle of less than 1:100. In this case, it is especially helpful if the duration of the measurement is minimal, for example the measurement of a color change of an indicator or sensor can be established with a measurement time of less than 10 ms, in particular less than 50 microseconds, or less than 1 microsecond.

In addition, the energy consumption can thus be reduced by means of a duty cycle, i.e. the sensor is operated cyclically but only during a 1/10 or 1/100 (or even shorter measurement intervals) of the time. If the measurement speed of the sensor is high (that is to say the time for a measurement is short), an exact or even highly exact measurement nevertheless takes place owing to the inertia with regard to changes in the air flow composition.

According to a further exemplary embodiment, the sensor module comprises at least one further sensor with sensor electronics. In particular, more than three sensors with corresponding sensor electronics can be provided, which are configured for measuring a value of an analysis parameter for the analysis of the air accompanying substances and/or of the air quality of the air, wherein in particular one sensor represents a combination sensor which is configured to determine multiple analysis parameters.

Energy-saving sensors are increasingly smaller and smaller. These increasingly miniaturized sensors allow two, three or more than three different sensors to be integrated in the filter module. In particular, it has been found that the use of combination sensors which determine multiple measurement variables with one sensor is particularly advantageous because only the air flow continuity at a specific location of the filter body has to be ensured for this purpose.

According to a further exemplary embodiment, the sensor comprises a movable component. Furthermore, certain sensors frequently use a movable component (e.g. a small fan which constantly regulates the throughflow quantity at the same level, deflected mirrors or filters for spectrometers or electromechanical distance changes [tunable frequency filters for spectroscopy on the basis of MEMS]). Precisely such components are particularly endangered with regard to pollution or bearing wear. The service life is therefore limited, and the time of the guaranteed exact measurements is significantly lower than the service life of the filter system. Due to the periodic filter module change (e.g. monthly, semi-annually, annually), the aging or the biasing (i.e. the continuous shifting of a measurement result over time) of the sensors starts again at ‘zero’. Various sensors also use an integrated controller. As a result, it is very easily possible to output a signal that a filter module exchange (due to aging or due to a detected disturbance) becomes due, or is pending within foreseeable time in the sense of preventive maintenance.

According to a further exemplary embodiment, the sensor electronics are adapted to store the measured analysis parameters, wherein the filter module in particular comprises a coupling element, which is mechanically and/or electrically coupled to the sensor electronics and is couplable to a terminal of the filter system. The coupling element is in particular configured such that a releasable coupling between the filter module and the terminal of the filter system is providable.

The coupling element is in particular configured such that upon insertion of the filter module into an operating position in the filter system, a coupling between the terminal of the filter system and the sensor electronics is automatically generatable, wherein the coupling element is in particular provided on an exhaust air side of the filter body. An optional controller integrated in the filter module can also store sensor values and subsequently forward them upon communication interruption or it becomes possible to read the data offline after the filter change. The data transmission can be carried out both wirelessly and wired (or in combination of both). For a wired (data, energy, configuration) electrical connection, the coupling element is advantageously used as an electrical plug-in connection, in particular an electrical connection, which is automatically unplugged and/or plugged in upon the filter module change. For the plug-in device does not become spoiled by loaded air (and thus unreliable), the connection is preferably attached on the exhaust air side of the filter.

The coupling element serves for signaling or electrical coupling between the sensor and devices of the filter system. The coupling element is in particular provided on the filter module, for example on the carrier frame of the filter module, such that in an operating position of the filter module in the filter system, a coupling with a respectively corresponding coupling element of the filter system is made possible. The coupling element can for example be an electrical plug.

By placing this plug-in connection in particular in the exhaust air region of the filter system, a pollution of the plug-in connection can be reduced or prevented.

According to a further exemplary embodiment, the sensor is configured to measure analysis parameters which determine an energy consumption and/or a CO2 footprint of the filter module and/or of the filter system 150, wherein the analysis parameters are in particular selected to determine a recommendation regarding filter replacement and/or filter cleaning, in particular that individual parameters are configurable in this case. A filter module requires increasingly more energy for the intended use with increasing occupancy, because a delta p or a pressure drop across the filter module increases as a result of the filter occupancy. On the basis of the data or analysis parameters, a recommendation regarding optimum filter module replacement time point (or cleaning time point) can be determined and communicated, preferably that individual parameters such as energy costs, savings potential, CO2 savings, CO2 certificate costs etc. are configurable or determinable in this case.

According to a further exemplary embodiment, the sensor is configured to measure analysis parameters for the analysis of fine dust,

wherein in particular the frequency of an occurrence of fine dust, in particular the analysis of the frequency of diameter classes of particles of the fine dust and/or of the composition of the fine dust, wherein the sensor is in particular configured to carry out the measurement in real time. A corresponding sensor for an analysis of the fine dust particles of the supply air can be attached on the supply air side of the filter body. In contrast to a pipe or channel, the cross section of the air guide is usually larger at locations of the filtering, which reduces the flow velocity at a given air throughput. A reduced flow velocity in turn leads to a more homogeneous air flow guidance and to less turbulent air turbulence (which for example force fine dust particles radially away). This makes it possible in an ideal manner to make measurements on the supply air side, which make a reference to the fine dust composition (diameter, quantity, substance analysis, etc.). The possibility according to the invention of external communication also permits real-time analyses of a fine dust load.

According to a further exemplary embodiment, the sensor is a dynamic pressure gauge and is in particular configured such that a static pressure upstream of the filter body and a static and dynamic pressure downstream of the filter body are measurable. If the velocity of the air flow is high enough, then the differential pressure between a normal pressure tap upstream of the filter body and a dynamic pressure pipe (or Pitot pipe) downstream of the filter can be measured. The pressure at the Pitot pipe is given by the sum of the static pressure and the dynamic pressure and is therefore higher than at the normal pressure tap upstream of the filter. This configuration creates an inverted or negative differential pressure across the filter and allows clogged supply lines or flap disturbances to be detected. This embodiment should be suitable in particular for retrofitting older installations. By using a controller in the filter module or filter system, it becomes possible to parameterize the response and/or limit values in the filter system from the outside.

The sensor can comprise, for example, a microphone and detect the noise level in a room and in particular the location of a noise source. By measurement and evaluation of the noise level in a room, it is possible to infer the number and intensity of speech-active persons in the room and the ventilation power of the fan unit can be adapted thereto via the control unit since the emission of aerosols by persons increases with the speech volume. In other words, the regulation of the ventilation power can thus be set via the noise level in the room. The more persons speak, or speak loudly, the more aerosols are emitted and the higher the ventilation power can be since then, for example, the additional sound of the devices, such as, for example, the fan unit, is not perceived and does not disturb. If one or more persons sit still in the room, the ventilation power drops because it must be quiet for concentrated work, wherein however also hardly any aerosols are emitted. According to a further exemplary embodiment, the filter module comprises a signal transmission unit which is configured for wireless or wired transmission of signals, wherein the signal transmission unit is in particular configured to transmit the data (of the sensor) by means of RFID, NFC, Bluetooth, WLAN or protocols of building control technology. A warning signal can be generated on the basis of these data by means of a control unit and/or a measure can be taken which in particular relates to a throughput through the filter module.

The signal transmission unit can, for example, an antenna or a conductor-based system which signals the readiness of the ventilation system to receive data from the filter module. Such data may relate not only to parameters relating to the air accompanying substances of the air, but also to information and details of the filter module. Thus, for example, depending on the performance of a filter module used, the air volume can be adapted by the filter module or the filter system. Furthermore, when a run time or occupation density of the filter module is exceeded, a signal can be emitted which can either be interpreted as a maintenance signal or can also be used as a control signal in order to reduce the air throughput quantity. An embodiment variant of the signal transmission unit can be an RFID transponder (which for example also comprises filter data in encrypted form). Furthermore, other communication mechanisms such as NFC, Bluetooth, WLAN etc. can also be used. For a wired communication, in addition to proprietary protocols, bus systems of building control systems (LON, EIB, etc.) are also available. By means of this mechanism, it is also possible to deliver a filter system in which functions are only enabled if a part of the unique ID belongs to the agreed delivery scope.

According to a further exemplary embodiment, the filter module comprises a receiving device which is configured for receiving a unique ID, wherein the unique ID comprises information regarding the location of use of the filter module. The receiving device is configured for reading the unique ID from a QR code, a barcode, an OCR font or an RFID tag. The receiving device is in particular configured for receiving the unique ID via NFC, Bluetooth, WLAN, proprietary protocols or protocols of building control systems, in particular LON or EIB, wherein the operation and/or the configuration of the filter module is adjustable based on the unique ID.

In a further particularly preferred embodiment, the unique ID comprises information regarding the installation location of the filter module in the filter system. This ID allows the operating parameters required for the specific operation to be preselected or stored data of a system configuration to be retrieved from a preconfigured operating mode of the filter system or of the filter module. In particular when using encrypted protocols, it is thus possible during the filter replacement to avoid a new configuration and to realize a ‘plug and play’ function. Corresponding data can be transmitted by the filter system or the filter module during the replacement or can be transferred via cloud. The transmission of the unique ID to the filter system can take place using mechanisms known to the person skilled in the art using QR code, barcode, OCR fonts (and their successors for machine-readable fonts), RFID, NFC, Bluetooth, WLAN, proprietary protocols or protocols of building control systems (LON, EIB, etc.). By means of this mechanism, it is also possible to deliver a filter system in which functions are only enabled if a part of the unique ID belongs to the agreed delivery scope.

According to a further exemplary embodiment, the filter body comprises a pocket filter or a hose filter. In a further particularly preferred embodiment, a filter system comprises multiple filter modules with pocket or hose filters. Instead of one of these pocket or hose filters, a functional unit can now be used for an additional function, such as for example as an energy supply unit or electrical supply unit. By means of the large structural volume thus present, it is possible for example to realize a plurality of additional functions. A life-long battery allows a simple retrofitting of existing filter modules by means of the solution according to the invention, without additional electrical and/or installation measures.

According to a further exemplary embodiment, the filter body comprises at least two fleece layers and a filter membrane arranged between the fleece layers, which are arranged in a layered manner one above the other in a layer composite, wherein in particular the middle filter membrane of the layer composite comprises a larger surface area than the two outer fleece layers. In particular according to an exemplary embodiment, a first direction (e.g. X direction) and a second direction (e.g. Y direction) are defined, which span a plane, wherein the middle filter membrane is corrugated with corrugation portions such that the corrugation portions are arranged one behind the other along a first direction. The corrugation portions run irregularly and asymmetrically with respect to one another, in particular within the plane. The filter body is arranged such that air can flow over the filter body along the first direction or along the second direction. For example, the x direction is the air inflow direction of the air and the corrugation portions run transversely to the first direction along the second direction. The asymmetry of the corrugation arrangement and shape can be used for vibration damping. Alternatively, the filter body can also be flown against in the Y direction and therefore parallel to the extent of the corrugations. The corrugation portions therefore form for example a shark skin-like riblet structure which brings about a reduction of the flow resistance. Depending on the entry conditions (inflow cross section, volume flow, depth of the filter material to be flowed through) into the filter body, one or other configuration can be particularly advantageous. The asymmetry of the corrugation arrangement can be achieved by a self-organizing compaction process in which the feed rate of the filter membrane is significantly higher than the feed rate of the two cover fleeces. The asymmetry of the corrugation arrangement arises as a result of thermal fixing of the three layers at a predetermined point in time. In addition to the advantages already described, this asymmetry has a stabilizing effect on deflections in the x-y plane.

According to a further exemplary embodiment, the filter body has a thickness of 2 mm to 10 mm, in particular of 3 mm to 7 mm, in the filter region. Additionally or alternatively, the number of corrugation portions is between 0.5 and 3 corrugations per cm. This allows a filter performance similar to a HEPA filter, but with a pressure drop in the region of a normal F7 filter (i.e. within the operating parameters of the solution according to the invention).

According to a further exemplary embodiment, the filter region is formed from a hydrophobic filter material. Furthermore, the filter region can be formed from natural fibers. The filter region can furthermore contain a polyolefin, in particular a polypropylene. In a further example, the filter region contains cellulose, cotton and/or hemp.

If the air flow to be filtered is loaded with a high aerosol load, known filters can have a tendency to soaking. On the one hand, this can statically increase the pressure drop across the filter, but also dynamically overcharge a subsequent volume flow regulation by means of VAV in the sense of its regulation speed due to the very rapidly changing pressure conditions. The solution according to the invention can solve this problem by a suitable choice of material of the filter material: either a hydrophobic material (for example a polyolefin, in particular polypropylene, which is substantially free of polar groups) or an absorbent material with a specific (for example low) tendency to swell (for example a natural fiber, in particular a cellulose fiber, cotton or hemp) is used. Thus, the tendency of filling filter openings with micro- or nanoscale water droplets is reduced. It has been found that the fungicidal, virucidal and bactericidal properties of hemp are favorable and make it an ideal filter constituent.

On the one hand, the air flow can be impeded by the insertion of the sensor with the sensor electronics, which leads to a larger pressure drop and thus to a higher energy consumption-on the other hand, this topic can be compensated for again by lowering the volume flow through the filter in the filter region. By means of the solution according to the invention, a filter region with an overdimensioned filter area (for example by wave-shaped integration of the filter membrane between two fleeces) can be created, which allows the volume flow per time and area to be correspondingly reduced. Thus, during operation, air volume quantities (m³) per hour and square meter of filter area of less than 450 m³, in particular less than 140 m³, preferably less than 85 m³, particularly preferably less than 50 m³ are possible. This leads at the same time to a less rapid occupation of the filter and as a result to less frequent filter changes or less frequent cleaning processes, which in turn reduces the operating resources.

According to a further exemplary embodiment, the filter region is formed from a hydrophobic filter material. Furthermore, the filter region can be formed from natural fibers. The filter region can furthermore contain a polyolefin, in particular a polypropylene. In a further example, the filter region contains cellulose, cotton and/or hemp.

If the air flow to be filtered is loaded with a high aerosol load, known filters can have a tendency to soaking. On the one hand, this can statically increase the pressure drop across the filter, but also dynamically overcharge a subsequent volume flow regulation by means of VAV in the sense of its regulation speed due to the very rapidly changing pressure conditions. The solution according to the invention can solve this problem by a suitable choice of material of the filter material: either a hydrophobic material (for example a polyolefin, in particular polypropylene, which is substantially free of polar groups) or an absorbent material with a specific (for example low) tendency to swell (for example a natural fiber, in particular a cellulose fiber, cotton or hemp) is used. Thus, the tendency of filling filter openings with micro- or nanoscale water droplets is reduced. It has been found that the fungicidal, virucidal and bactericidal properties of hemp are favorable and make it an ideal filter constituent.

According to a further exemplary embodiment, the filter module comprises a support frame, which can be fastened in particular to a housing of a filter system (for example exchangeably). Furthermore, the filter module comprises a filter frame, to which the filter body can be fastened, wherein the filter frame is arranged in particular exchangeably on the support frame. Additionally or alternatively, the filter module comprises a sensor frame, to which at least the sensor (or the further sensors) can be fastened, wherein the sensor frame is arranged in particular exchangeably on the support frame. The support frame represents the structure which can transmit holding forces from the filter module and from the sensors. The support frame is fastened in particular to a housing of the filter system. The support frame can be arranged for example exchangeably in the filter system, for example in the housing of the filter system, with the result that the entire filter module is exchangeable.

The support frame represents a fastening device for the filter frame and the sensor frame. The filter frame and/or the sensor frame can be fastened exchangeably to the support frame. Therefore, a modular system can be provided in which elements to be serviced, such as for example a filter module or a sensor, have to be replaced without changing the entire filter module. If for example the filter body is clogged or occupied, the filter frame can be detached from the support frame. A new filter body with a corresponding filter frame can subsequently be fastened exchangeably to the support frame. Correspondingly, the sensor frame with the sensors can also be exchanged if a defect of a sensor is detected. The exchange of the sensor frame can also be carried out without an exchange of the filter frame. Furthermore, a sensor can also be fastened to the filter frame, with the result that said sensor can be exchanged together with an exchange of the filter frame.

Therefore, a sustainable and efficient exchange or changing system is created, with the result that only the components to be serviced or the defective components, such as for example an occupied filter body or a defective sensor, can be exchanged without the remaining components likewise having to be exchanged.

The sensor frame and the filter frame can be fastened to the support frame for example by means of a screw connection. In this case, a common fastening means, such as for example a screw, can simultaneously fasten the sensor frame and the filter frame to the support frame. Furthermore, further releasable fastening means are also possible, such as for example a latching connection with latching element, wherein a corresponding sensor frame or filter frame can be loaded into the support frame. Hook-and-loop fastener connections can likewise be used to connect the filter frame and/or the sensor frame to the support frame.

In particular, the sensor frame is arranged between the support frame and the filter frame. The sensor can be fastened for example to the frame itself of the sensor frame. Furthermore, a spacer element can project from the sensor frame into the center of the sensor frame in order to space the sensor from an edge of the sensor frame.

According to a further exemplary embodiment, the filter module comprises a weighing device, which is configured to weigh the filter occupancy, in particular such that a measured value falsification by the pressure of the air flowing through the system can be compensated. With corresponding additional mechanisms, a compensation of the measured value falsification by the pressure of the air resistance can be achieved during operation of the filter system. This also allows the establishment of a high filter occupancy for an operating mode of the filter system at a low volume flow, which does not lead to triggering of the differential pressure monitoring of the filter in the case of customary filter monitoring. In particular, the weighing device can have a ground contact in the installed state of the filter module in the housing of the filter system and therefore introduce the weight force of the filter module into the ground. As a result, a weight measurement of the filter module can be carried out.

According to a further exemplary embodiment of the filter system, the control unit comprises a visualization unit, which is configured to visualize the air quality and the analysis of the air particles, in particular in a location-dependent manner at the location of the corresponding filter system. Furthermore, the control unit is in particular configured to generate an action recommendation based on the air quality and the analysis of the air accompanying substances.

In an especially preferred embodiment, multiple filter modules are operated with one another in such a way that they clean the air from an individual building section. These filter modules have corresponding additional equipment, which can exchange data regarding air details and thus air measurement data can be visualized at at least one point or actions dependent thereon can be initiated. Here, both filters of a primary ventilation among themselves and filter systems of secondary ventilations among themselves, as well as all of them, can form a network with one another and interact with one another. In particular, a location-dependent air quality can thus be visualized, an action recommendation can be given or a measure can be initiated (e.g. “meeting room 2 has bad air” or “air quality low, please raise a level of the fan”).

According to a further exemplary embodiment, the filter system comprises a flow controller, for example comprising a fan or other flow generators. A flow velocity of the air through the filter body and an air pressure of the air at the supply air side of the filter body are adjustable by means of the flow controller. The flow controller is configured to adjust a pressure drop difference from a pressure drop between the supply air side and the exhaust air side in the filter region by means of mechanical and/or mechatronic flow controller systems in such a way that a constant volume flow through the filter region is adjustable, in particular based on a subsequent adaptation based on measurement data.

The flow controller systems comprise, for example, a mechanical cross-sectional change of the inlet into the filter body, wherein the pressure drops in the filter region are taken into account in such a way that the air flow portion at the sensor remains similar to and representative of the air flow portion in the filter region. A pressure drop at the sensor can be adjusted adaptively, depending on the pressure drop in the filter region.

The control unit can take into account, for example, that a pressure drop at the sensor is taken into account, for example, in the measurement data of the sensor. Furthermore, the analysis can function over a wide pressure drop range in that, for example, the pressure difference is taken into account in the post-processing of the measurement data by the control unit (e.g. by permanently recording the pressure difference and taking these data into account in the evaluation).

According to a further exemplary embodiment, the flow controller is configured in such a way that a volume flow per square meter of filter area of the filter module is adjustable below 140 m³/h, in particular below 85 m³/h, preferably below 50 m³/h.

According to a further exemplary embodiment, the flow controller is configured in such a way that the velocity of the volume flow is adjustable in the range of 0.1 m/s to 5 m/s, in particular 0.2 m/s to 3.4 m/s, in particular between 0.3 m/s to 2.8 m/s, and/or the pressure drop over the filter module is adjustable in at least one operating mode below 450 Pa, in particular below 250 Pa, preferably below 150 Pa. If the filter system is operated within these characteristic values, it can be ensured by means of the measures proposed in the invention (or a combination thereof) that the sensor comes into contact with more than 90% with the same accompanying substances and accompanying substance quantities as the average value in the entire air flow, as mentioned. The composition of the air flow at the sensor also changes only insignificantly in the case of a pressure drop variation of 50 Pa to 450 Pa. The solution according to the invention is suitable in particular for filter systems which have a pressure drop over the filter system of below 2500 Pa, since the optimizations of the flow resistances according to the invention are only then taken into account. Especially good results are achieved when, in one operating mode, there is a pressure drop of less than 450 Pa, in particular less than 250 Pa, preferably less than 150 Pa.

According to a further exemplary embodiment, the filter module further comprises a receiving device with a receiving region for receiving the filter module, wherein at least one of the receiving regions is configured to receive a filter module or a functional unit, wherein the functional unit in particular comprises a power supply device for supplying the sensor and the sensor electronics with electrical energy. The receiving device of the filter system can be formed for example with guide rails which are arranged on a housing of the filter system and along which a filter module or a functional unit can be pushed into the operating position within the filter system. Furthermore, for example, releasable fastening means, such as for example screws or clamping closures, can be provided as receiving device in order to arrange the filter module or the functional unit modularly and exchangeably in the filter system. The functional unit can for example represent a supply unit for electrical energy, such as for example a rechargeable battery pack.

According to a further exemplary embodiment, the filter module further comprises a storage module for storing data, wherein the data are indicative of the values of the analysis parameters, in particular at least one analysis parameter selected from the group consisting of air throughput through the filter system, temperature of the air which is flowing through, pressure of the air which is flowing through, in particular absolute pressure and/or differential pressure, filter occupancy of the filter body, humidity of the air which is flowing through, aerosol load of the air which is flowing through or the PM content, wherein the storage module is configured such that the storage module is couplable to a reading device outside the filter system for reading the data.

Particularly in the case of especially demanding operating conditions, it can be of interest to store individual detection details in a storage module and to parameterize the data. On the one hand, this relates to details of the measuring method, in particular temporal measured value changes and for example the number of exceedances of limit values, on the other hand, this also relates to the storage and subsequent comparison of a plurality of different parameters, for example air throughput, temperature, pressure (in particular absolute pressure and/or differential pressure), filter occupancy, humidity, aerosol load, PM content [in particular also how much of which diameter class]. Such a data record can then in turn be transmitted by means of a signal transmission unit, or stored in the storage module and read after the end of the filter module service life. Furthermore, use data of the filter module, such as for example use duration and use intensity (for example of the flow controller), can be stored in the storage module. Based on these data, for example consumption bills relating to the use of the filter module can be created which can be billed to the user of the filter module. The generation of consumption bills can be used in particular in rental objects, wherein each tenant receives a consumption bill assigned to him as a function of the use of the filter module.

According to a further exemplary embodiment, the filter module further comprises a covering device which is configured to selectively cover an air inlet on a supply air side of the filter system, wherein the covering device is in particular configured such that upon a change of the filter module, the covering device automatically covers the air inlet.

Since high-quality sensorics are installed in the filter module by the solution according to the Invention, which sensorics can at best be recycled, in a further embodiment a protective function for recontamination of the environment or sensors by foreign substances embedded in the filter or adhering to the filter is installed, which protective function can be activated upon a change of the filter, in particular which protective function is carried out automatically. This can be for example in the case of a pocket filter a shutter-like or slider-like covering device which covers the filter inlet or outlet of the filter system upon withdrawal of the filter module. The covering device can also be attached temporarily by means of adhesive or hook-and-loop connections during an exchange.

It is pointed out that the embodiments described here represent only a limited selection of possible embodiment variants of the invention. Thus, it is possible to combine the features of individual embodiments with one another in a suitable manner, so that for the person skilled in the art with the embodiment variants explicit here, a multiplicity of different embodiments are to be regarded as obviously disclosed. In particular, some embodiments of the invention are described with device claims and other embodiments of the invention are described with method claims. However, the person skilled in the art will immediately become clear upon reading this application that, unless explicitly stated otherwise, in addition to a combination of features which belong to a type of subject matter of the invention, an arbitrary combination of features which belong to different types of subject matter of the invention is also possible.

### BRIEF DESCRIPTION OF THE DRAWINGS

In the following, exemplary embodiments are described in more detail with reference to the attached drawings for further explanation and for better understanding of the present invention. In the drawings:

FIG. 1 shows a filter system with a filter module according to an exemplary embodiment.

FIG. 2 shows a schematic representation of a filter material for the filter body according to an exemplary embodiment.)

FIG. 3 shows a schematic representation of wave shapes of the filter material according to an exemplary embodiment.

FIG. 4 shows a schematic representation of a filter module with a dynamic pressure gauge according to an exemplary embodiment.

FIG. 5 shows a schematic representation of a filter system with a filter module and multiple filter bodies according to an exemplary embodiment.

FIG. 6 shows a perspective representation of a modular filter module according to an exemplary embodiment.

FIG. 7 shows a side view of the modular filter module from FIG. 6 according to an exemplary embodiment.

### DETAILED DESCRIPTION OF EXEMPLARY EMBODIMENTS

Same or similar components in different figures are provided with identical reference numerals. The representations in the figures are schematic.

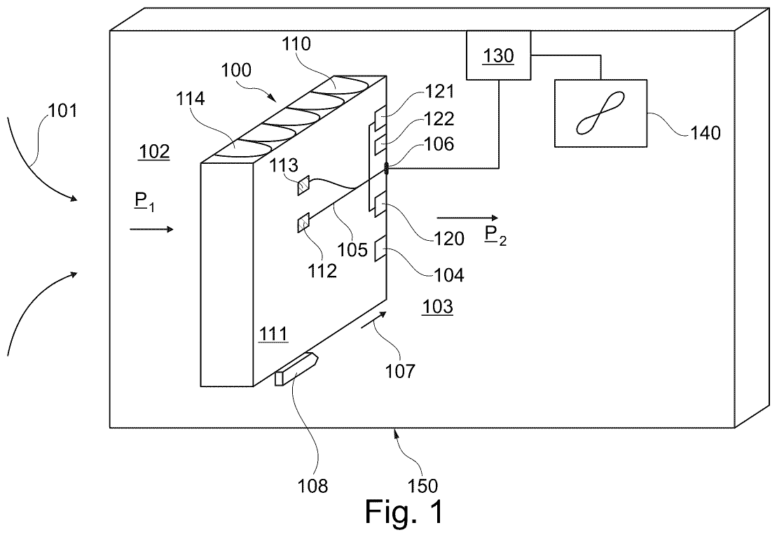

FIG. 1 shows a filter system 150 with a filter module 100 according to an exemplary embodiment. The filter system 150 comprises a control unit 130 and at least one filter module 100, wherein the at least one filter module 100 is configured for the exchange of analysis data which are required for the support of the analysis of the air accompanying substances and/or of the air quality.

The filter module 100 comprises a filter body 110, which filters the air 101 which is flowing through from air accompanying substances, and at least one sensor 112 or further sensors 113 with sensor electronics. The sensor 112 is configured for measuring a value of an analysis parameter for the analysis of the air accompanying substances and/or of the air quality of the air 101. The filter body 110 and the sensor 112 are configured and arranged with respect to one another in such a way that, in the case of an air flow through the filter module 100, the value of the analysis parameter which is measured with the sensor 112 corresponds to more than 90% to the value of the analysis parameter which is averaged over the entire air flow. The filter body 110 is configured in such a way that a pressure drop over the filter body 110 is less than 2500 Pascal in the case of an air volume per square meter of filter area and hour of less than 600 m³/(m²×h) and a velocity of the volume flow is in the range of 0.1 m/s to 5 m/s.

The filter system 150 comprises a housing, in which a filter module 100 is arranged. Here, a filter module 100 according to the invention is arranged exchangeably in the filter system 150. For example, corresponding guide rails can be provided, along which the filter module can be pushed into the insertion direction 107 as far as the operating position within the filter system 150.

The filter system 150 comprises a flow controller 140, for example comprising a fan or other flow generators. A flow velocity of the air 101 through the filter body 110 and an air pressure of the air 101 at the supply air side 102 of the filter body 110 are adjustable by means of the flow controller 140. The flow controller 140 is configured to adjust a pressure drop difference from a pressure drop between the pressure p1 of the supply air side 102 and the pressure p2 of the exhaust air side 103 in the filter region 111 and at the sensors 112, 113, in particular by means of mechanical and/or mechatronic flow controller systems, in such a way that a constant volume flow through the filter region 111 and at or through the sensors 112, 113 is adjustable, in particular based on a subsequent adaptation based on measurement data.

The filter module 100 comprises a two-dimensional filter material, which is fixed in a receiving device, for example a circumferential support frame. The filter module 100 can be formed as a pocket filter, wherein a multiplicity of pockets 114 of filter material are fastened in the support frame and the air flow is introduced into the pockets 114 in order to filter the inflowing air 101.

The filter module 100 comprises in particular the filter region 111 which assumes the function of filtering the air 101. The sensors 112, 113 comprise a sufficient distance from the support frame of the filter module 100 or to the edge of a flow channel, in which the filter module 100 is arranged in the filter system 150, in order thus to avoid edge flow properties which cause a different composition of the air particles of the air or a different pressure drop range of the air 101, relative to a, for example central, filter region.

The sensors 112, 113 serve for measuring at least one parameter of the air accompanying substances and/or of the air quality of the air 101. The direct measurement of foreign substances or groups of foreign substances in the air flow can be provided by means of one of the sensors 112, 113. The sensor 112, 113 can comprise a MEMS sensor. Furthermore, one of the sensors 112, 113 can in particular be configured such that they can be used for a Fourier transform infrared spectrometer analysis FTIR and/or a near infrared spectroscopy analysis. The sensors 112, 113 can for example form a resistance sensor for measuring the air accompanying substances and/or the air quality. In this case, for example, a trigger substance can additionally be used in order to measure the presence of certain foreign substances in the air 101.

The filter module 100 comprises a communication unit 122 for the communication of data relating to the air accompanying substances and/or the air quality to a control unit 130 of the filter system 150, in particular for controlling the filter module 100. The communication unit 122 is configured to transmit information relating to the air accompanying substances and/or the air quality to the control unit 130 or likewise control signals which can be created based on measured parameters in order for example to generate control signals relating to indication signals (alarm signals) or air flow control signals to which the control unit 130 transmits.

The filter module 100 further comprises a coupling element 106, which is mechanically and/or electrically coupled to the sensors 112, 113 and is couplable to a terminal of a filter system 150. The coupling element 106 is in particular configured such that a releasable coupling between the sensors 112, 113 and the terminal of the filter system 150 is providable. Furthermore, the coupling element 106 is in particular configured such that upon insertion of the filter module 100 into an operating position in the filter system 150, a coupling between the terminal of the filter system 150 and the sensors 112, 113 is automatically generatable. The coupling element 106 is provided on an exhaust air side 103 of the filter body 110. The coupling element 106 is in particular provided on the filter module 100, for example on the carrier frame of the filter module 100, such that in an operating position of the filter module 100 in the filter system 150, a coupling with a respectively corresponding coupling element of the filter system 150 is made possible.

The filter module 100 further comprises a weighing device 108, which is configured to weigh the filter occupancy, in particular such that a measured value falsification by the pressure of the air 101 flowing through the system can be compensated. The weighing device 108 has a ground contact in the installed state of the filter module 100 in the housing of the filter system 150 and therefore introduce the weight force of the filter module 100 into the ground. As a result, a weight measurement of the filter module 100 can be carried out.

The filter module 100 comprises a receiving device 120 which is configured for receiving a unique ID, wherein the unique ID comprises information regarding the location of use of the filter module 100. The receiving device 120 can be configured for reading the unique ID from a QR code, a barcode, an OCR font or an RFID tag. Furthermore, the receiving device 120 can be configured for receiving the unique ID via NFC, Bluetooth, WLAN, proprietary protocols or protocols of building control systems, in particular LON or EIB. The operation and/or the configuration of the filter module 100 is adjustable based on the unique ID. For example, the unique ID comprises information regarding the installation location of the filter module 100 in the filter system 150. This ID allows the operating parameters required for the specific operation to be preselected or stored data of a system configuration to be retrieved from a preconfigured operating mode of the filter system 150 or of the filter module 100.

The filter module 100 further comprises a transmitting device 121 for transmitting filter-body-related data, wherein the transmitting device 121 is configured to transmit the data by means of RFID, NFC, Bluetooth, WLAN or protocols of building control technology. A warning signal can be generated on the basis of these data by means of the control unit 130 and/or a measure can be taken which in particular relates to a throughput through the filter module 100.

The transmitting device 121 can, for example, an antenna or a conductor-based system which signals the readiness of the ventilation system or filter system 150 to receive data from the filter module 100. Such data may relate not only to parameters relating to the air accompanying substances of the air 101, but also to information and details of the filter module 100. Thus, for example, depending on the performance of a filter module 100 used, the air volume can be adapted by the filter module 100 or the filter system 150. Furthermore, when a run time or occupation density of the filter module 100 is exceeded, a signal can be emitted which can either be interpreted as a maintenance signal or can also be used as a control signal in order to reduce the air throughput quantity. The sensor 112 can for example be supplied with electrical energy by an electrical supply unit 104. Corresponding signals can for example be transmitted between the sensor 112, the communication unit 122, the transmitting device 121 and the receiving device 120 by means of signal connections 105.

The control unit 130 can comprise a (possibly locally remote) visualization unit, which is configured to visualize the air quality and the analysis of the air particles or air accompanying substances, in particular in a location-dependent manner at the location of the corresponding filter system 150. Furthermore, the control unit 130 is in particular configured to generate an action recommendation based on the air quality and the analysis of the air accompanying substances.

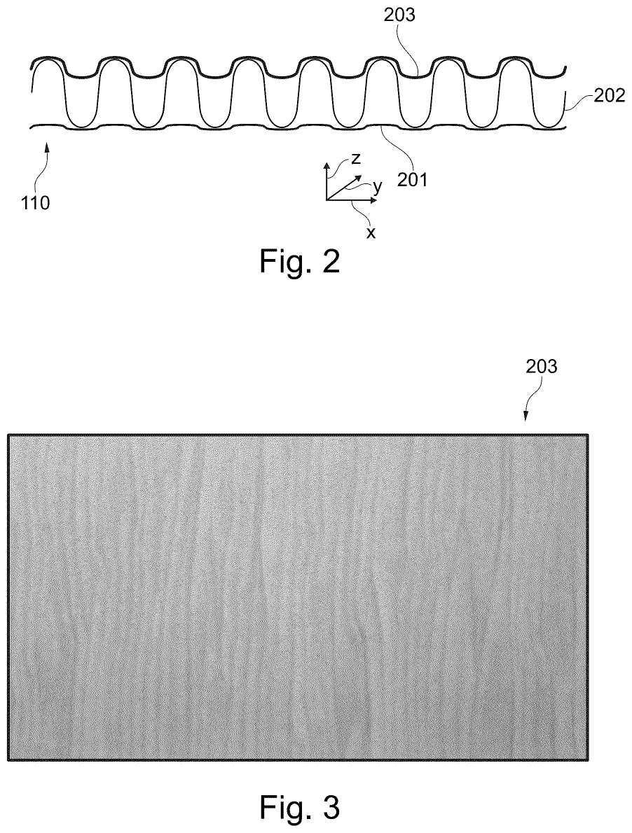

FIG. 2 shows a schematic representation of a filter material for the filter body 110 according to an exemplary embodiment. The filter body 110 comprises in particular in the filter region 111 multiple filter layers, which are arranged one behind the other in the flow direction of the air 101 through the filter, wherein in particular the first filter layer facing the supply air side 102 filters more coarsely than at least one of the second filter layers following the subsequent first filter layer in the flow direction. Therefore, coarser particles can initially be filtered, while smaller particles flow through the first layers and are only filtered out later in the case of the fine layers.

The filter body 110 or a layer comprises, in particular in the filter region 110, a fleece as filter material, wherein the fleece comprises in particular an entire layer or a multiplicity of layers.

The filter body 110 comprises at least two fleece layers 201, 203 and a filter membrane 202 arranged between the fleece layers, which are arranged in a layered manner one above the other in a third direction z in a layer composite, wherein in particular the middle filter membrane 202 of the layer composite comprises a larger surface area than the two outer fleece layers 201, 203.

The middle filter membrane 202 comprises corrugation portions, which are arranged one behind the other along a first direction x.

FIG. 3 shows a schematic representation of wave shapes of the filter material according to an exemplary embodiment. The corrugation portions run irregularly and asymmetrically with respect to one another, in particular within the plane. The filter body 110 is arranged such that air can flow over the filter body 110 along the first direction x or along the second direction y. For example, the x direction is the air inflow direction of the air 101 and the corrugation portions run transversely to the first direction x along the second direction y. The asymmetry of the corrugation arrangement and shape can be used for vibration damping.

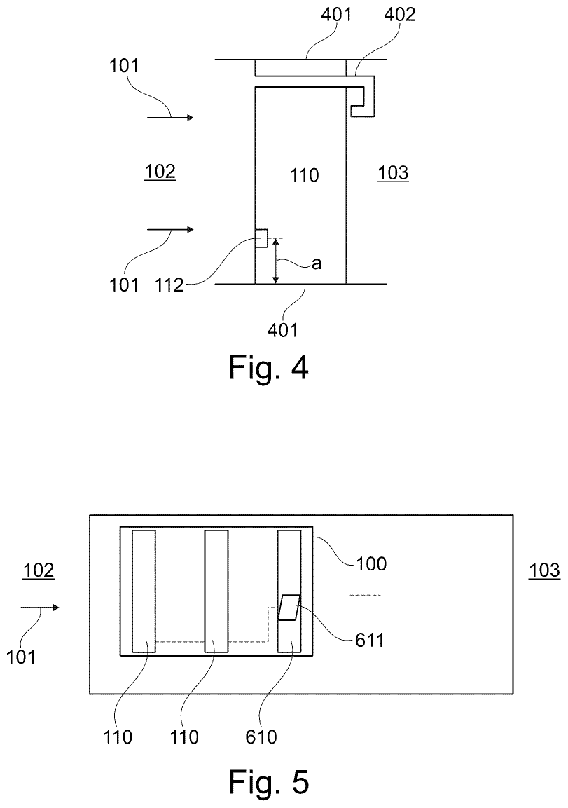

FIG. 4 shows a schematic representation of a filter module 100 with a dynamic pressure gauge 402 as a sensor according to an exemplary embodiment. The dynamic pressure gauge 402 is configured such that a static pressure upstream of the filter body 110 on the supply air side 102 and a static and dynamic pressure downstream of the filter body 110 on the exhaust air side 103 are measurable. If the velocity of the air flow is high enough, then the differential pressure p1-p2 between a normal pressure tap upstream of the filter body 110 (pressure p1) and a dynamic pressure pipe (or Pitot pipe) downstream of the filter body 110 (pressure p2) can be measured. The pressure at the Pitot pipe is given by the sum of the static pressure and the dynamic pressure and is therefore higher than at the normal pressure tap upstream of the filter body 110. This configuration creates an inverted or negative differential pressure across the filter body 110 and allows clogged supply lines or flap disturbances to be detected.

Furthermore, a sensor 112 is shown, which is arranged in particular at a distance a of more than 0.5 cm from the edge or support frame of the filter body 110, which acts as an outer air flow boundary 401, so that no edge effects with air flow turbulences occur at the sensor 112.

FIG. 5 shows a schematic representation of a filter system 150 with a filter module 100 and multiple filter bodies 110, 610 according to an exemplary embodiment. The filter bodies 110, 610 consist for example in each case of pocket filters or a hose filter, wherein at least one filter body 610 consists for example exclusively of a functional module which is configured for integrating additional functions in addition to the filtering. For example, at least one filter body 610 consists exclusively of a power supply unit 611, which is in particular designed such that a power supply is providable for a predetermined service life of the filter module 100. The filter module 100 comprises in the exemplary embodiment corresponding filter bodies 110, 610 arranged serially one behind the other.

The energy or power generation unit 611 is configured for example to obtain energy by means of the air flow through the filter module 100 and/or by electromagnetic waves, which energy is used in particular for operating the sensors 112, 113.

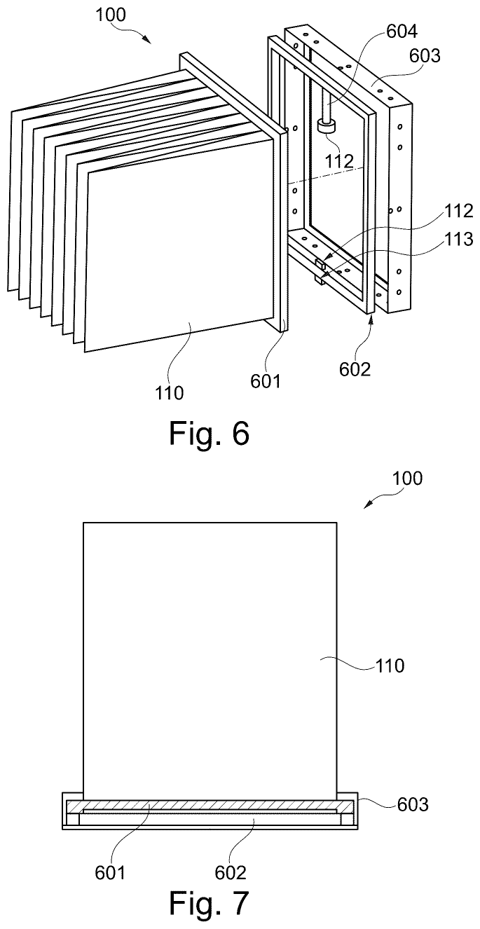

FIG. 6 and FIG. 7 show a schematic representation of a modular filter module 110 according to an exemplary embodiment. The filter module 100 comprises a support frame 603, which can be fastened in particular to a housing of a filter system 150. The support frame 603 can be arranged exchangeably on a sensor frame 602, to which the sensors 112, 113 are fastened. In addition, a filter frame 601, to which the filter body 110 is fastened, can be fastened exchangeably to support frame 603 and/or the sensor frame 602.

The support frame 603 represents a fastening device for the filter frame 601 and the sensor frame 602. The filter frame 601 and/or the sensor frame 602 can be fastened exchangeably to the support frame 603. If the filter body 601 has to be exchanged, the filter frame 601 can be detached from the support frame 603. The sensor frame 602 can remain fastened to the support frame 603. Furthermore, the sensor frame 602 with the sensors 112, 113 can also be exchanged without an exchange of the filter frame 601 having to be carried out.

The sensor frame 602 is arranged for example between the support frame 603 and the filter frame 601. The sensor 112 can be fastened for example to the sensor frame 302. Furthermore, a spacer element 604, such as for example a support rod, can project from the sensor frame 602 into the center in order to space the sensor 112 from an edge of the sensor frame 602.

In addition, it should be noted that “comprising” does not exclude any other elements or steps and “a” or “an” does not exclude a multiplicity.

Furthermore, it should be pointed out that features or steps which have been described with reference to one of the above exemplary embodiments can also be used in combination with other features or steps of other exemplary embodiments described above. Reference signs in the claims are not to be regarded as a restriction.

### LIST OF REFERENCE SIGNS

100 Filter module
101 Air
102 Supply air side
103 Exhaust air side
104 Electrical supply unit
105 Signal connection
106 Coupling element
107 Insertion direction
108 Weighing device
110 Filter body
111 Filter region
112 Sensor
113 Further sensor
114 Pocket
120 Receiving device
121 Signal transmission unit
122 Communication unit
130 Control unit
140 Flow controller
150 Filter system
201 Outer fleece layer
202 Filter membrane
203 Outer fleece layer
401 Outer air flow boundary
402 Dynamic pressure gauge
601 Filter frame
602 Sensor frame
603 Support frame
604 Spacer element
610 Further filter body
611 Power supply unit
a Distance air flow boundary
x First direction
y Second direction
z Third direction
p1 Pressure supply air side
p2 Pressure exhaust air side

1. A filter module for filtering air of at least a part of a building or of air of an exhaust air purification unit of a production process, wherein the filter module comprises:
a filter body which filters the air which is flowing through from air accompanying substances, and
at least one sensor with sensor electronics, which is configured for measuring a value of an analysis parameter for the analysis of at least one of the air accompanying substances and of the air quality of the air,
wherein the filter body and the sensor are configured and arranged with respect to one another in such a way that, in the case of an air flow through the filter module, the value of the analysis parameter which is measured with the sensor corresponds to more than 90% to the value of the analysis parameter which is averaged over the entire air flow,
wherein the filter body is configured in such a way that a pressure drop over the filter body is less than 2500 Pascal in the case of an air volume per square meter of filter area and hour of less than 600 m³/(m²×h) and a velocity of the volume flow is in the range of 0.1 m/s to 5 m/s.

2. The filter module according to claim 1,
wherein the sensor is configured to measure an analysis parameter of at least one of a gas, a liquid and a solid as an air accompanying substance and an air constituent,
wherein the analysis parameter defines at least one of the chemical and physical properties of the air accompanying substance, with the result that at least one of an energy consumption, a savings potential, an air load and a CO₂ footprint can be determined taking into account the analysis parameters.

3. The filter module according to claim 1,
wherein the sensor is adapted to measure at least one analysis parameter relating to the temperature of the air, the humidity of the air, the flow rate of the air, the air quantity of the air through which air can flow, the dew point of the air, a proportion of an air accompanying substance in the air and an analysis parameter of the air accompanying substance.

4. The filter module according to claim 1,
wherein the filter material of the filter body comprises a fleece,
wherein the fleece is configured with one layer or with multiple layers.

5. The filter module according to claim 1,
wherein the filter module is exchangeably arrangeable in a filter system,
wherein the filter module comprises an information element which emits a signal regarding the due time of an exchange of the filter module.

6. The filter module according to claim 1,
wherein the sensor is arranged on an exhaust air side of the filter body.

7. The filter module according to claim 1, comprising at least one of the following features:
i) wherein the sensor can be operated discontinuously;
ii) further comprising
at least one further sensor with sensor electronics, which is configured for measuring a value of an analysis parameter for the analysis of at least one of the air accompanying substances and the air quality of the air;
iii) wherein the sensor comprises a movable component.

8-9. (canceled)

10. The filter module according to claim 1,
wherein the sensor electronics are adapted to store the measured analysis parameters.

11. The filter module according to claim 1, comprising at least one of the following features:
i) wherein the sensor is placed more than 0.5 cm from the outer air flow boundary of the filter body;
ii) wherein the sensor is configured to measure analysis parameters which determine at least one of an energy consumption and a CO2 footprint of at least one of the filter module and the filter system;
iii) wherein the sensor is configured to measure analysis parameters for the analysis of fine dust;
iv) at least one of wherein the sensor is a dynamic pressure gauge, and
wherein the sensor comprises a microphone which is configured to detect the noise level in a room such that the number and intensity of speech-active persons in the room can be determined by means of measurement and evaluation of the noise level in the room;
v) further comprising
a signal transmission unit which is configured for wireless or wired transmission of signals,
wherein at least one of a warning signal can be generated on the basis of these data by means of a control unit and a measure can be taken;
vi) further comprising
a receiving device which is configured for receiving a unique ID,
wherein the unique ID comprises information regarding the location of use of the filter module,
at least one of wherein the receiving device is configured for reading the unique ID from a QR code, a barcode, an OCR font or an RFID tag, and
wherein the receiving device is configured for receiving the unique ID via NFC, Bluetooth, WLAN, proprietary protocols or protocols of building control systems,
wherein at least one of the operation and the configuration of the filter module is adjustable based on the unique ID;
vii) wherein the filter body comprises a pocket filter or a hose filter.

12-17. (canceled)

18. The filter module according to claim 1,
wherein the filter body comprises at least two fleece layers and a filter membrane arranged between the fleece layers, which are arranged in a layered manner one above the other in a layer composite.

19. The filter module according to claim 18,
wherein a first direction and a second direction span a plane,
wherein the middle filter membrane is corrugated with corrugation portions such that the corrugation portions are arranged one behind the other along a first direction,
wherein the corrugation portions run irregularly and asymmetrically with respect to one another, and
wherein the filter body is arranged such that air can flow over the filter body along the first direction or along the second direction.

20. The filter module according to claim 19,
at least one of wherein the filter body has a thickness of 2 mm to 10 mm, and
wherein the number of corrugation portions is between 0.5 and 3 corrugations per cm.

21. The filter module according claim 1, comprising at least one of the following features:
i) at least one of wherein the filter body is formed from a hydrophobic filter material, and
wherein the filter body is formed from natural fibers, and
wherein the filter body contains a polyolefin, and
wherein the filter body contains at least one of cellulose, cotton and hemp;
ii) further comprising
a support frame, which can be fastened to a filter system,
a filter frame, to which the filter body can be fastened,
at least one of wherein the filter frame is arranged on the support frame, and
a sensor frame, to which at least the sensor can be fastened,
wherein the sensor frame is arranged on the support frame;
iii) further comprising
a weighing device, which is configured to weigh the filter occupancy;
iv) wherein the filter body comprises a pocket filter, a cartridge filter, a hose filter, a candle filter, a compact filter or a HEPA filter.

22-24. (canceled)

25. A filter system, comprising:
a control unit, and
at least one filter module according to claim 1,
wherein the at least one filter module is coupled to the control unit for the exchange of analysis data relating to the analysis of at least one of the air accompanying substances and of the air quality of the air.

26. The filter system according to claim 25,
wherein the control unit has a visualization unit, which is configured to visualize the air quality and the analysis of the air accompanying substances.

27. The filter system according to claim 25, further comprising:
a flow controller,
wherein a flow velocity of the air through the filter body and an air pressure of the air at the supply air side of the filter body are adjustable by means of the flow controller,
wherein the flow controller is configured to adjust a pressure drop difference from a pressure drop between the supply air side and the exhaust air side of the filter body.

28. The filter system according to claim 27,
wherein the flow controller is configured in such a way that a volume flow per square meter of filter area of the filter module is adjustable below 140 m³/h.

29. The filter system according to claim 28,
wherein the flow controller is configured in such a way that at least one of the velocity of the volume flow is adjustable in the range of 0.2 m/s to 3.4 m/s, and the pressure drop over the filter module is adjustable in at least one operating mode below 450 Pa.

30. The filter system according to claim 25, comprising at least one of the following features:
i) further comprising
a receiving device which comprises receiving regions for receiving the filter module;
wherein at least one of the receiving regions is configured to receive a filter module or a functional unit;
ii) further comprising
a storage module for storing data,
wherein the data are indicative of the values of the analysis parameters,
wherein the storage module is configured such that the storage module is couplable to a reading device outside the filter system for reading the data;
iii) further comprising
a covering device which is configured to selectively cover an air inlet on a supply air side of the filter system.

31-32. (canceled)

33. A method for filtering air of at least a part of a building or of air of an exhaust air purification unit of a production process with a replaceable filter module according to claim 1.
